(19) United States
(12) Patent Application Publication
(10) Pub. No.: US 2006/0281676 A1
(43) Pub. Date: Dec. 14, 2006

(54) Agent and method for the treatment and prevention of tse and method for the production of said agent

(76) Inventors: Dieter Willbold, Aldenhoven (DE); Jorg Smolinski, Baruth (DE); Karen Olga Hanel, Baesweiler (DE); Michael Andreas Wolff, Dusseldorf (DE); Detlev Riesner, Dusseldorf (DE); Carsten Korth, Dusseldorf (DE)

Correspondence Address: THE FIRM OF KARL F ROSS, 5676 RIVERDALE AVENUE, PO BOX 900, RIVERDALE (BRONX), NY, 10471-0900, US

(21) Appl. No.: 10/570,233

(22) PCT Filed: Aug. 4, 2004
(86) PCT No.: PCT/DE04/01738

§ 371 Date: Apr. 28, 2006

(30) Foreign Application Priority Data
Aug. 29, 2003 (DE) 103 40 260.8

(51) Int. Cl. A61K 38/17; C07K 14/47; C07H 21/04; C12P 21/06

(52) U.S. Cl. 514/12; 530/324; 435/69.1; 435/320.1; 435/325; 536/23.5

(57) ABSTRACT

The invention relates to an agent and/or pharmaceutical active ingredient and to a method for the treatment, prevention and diagnosis of TSE, and to a method for the production of an agent and/or pharmaceutical active ingredient. According to the invention, peptides and/or nucleotide sequences coding for said peptides are provided as disclosed in the sequence protocols 127 and partial sequences thereof are disclosed. Treatment or prevention of the TSE disease, for example, in humans with Jacob Creutzfeldt syndrome, kuru, Gertmann Sträussler-Scheinker syndrome and FFI (fatal familial insomnia) can be carried out with the aid of the inventive peptides. TSE diseases are also known in animals, such as Scrapie in sheep, BSE in cows and CWD in wild animals.

The invention relates to a method and an agent for the treatment and for the prevention of TSE and to a method of making the treatment agent.

The occurrence of transmissible spongiform encephalopathy (TSE) was originally thought to have a connection with prions in the results of early medical research. The illness was characterized by a sponge-like change in the brain structure. As the main source of the pathology, prions were identified. Prions consist predominantly of prion protein (PrP) in a pathogenic conformation (PrP^{Sc}). Healthy organisms also produce prion proteins, although in a nonpathogenic conformation (PrP^{c}) which is not dangerous. PrP^{c} and PrP^{Sc} have the same aminoacid sequence but have a significant difference in their secondary structures. PrP^{c} contains a relatively high α-helical component and practically no β-folded sheet elements. PrP^{Sc}, however, has a higher β-folded sheet proportion and a reduced α-helix proportion in a secondary structure which differs from that of PrP^{c}. As a consequence, PrP^{c} and PrP^{Sc} are structural isomers of one another. In addition, PrP^{Sc}, by contrast to PrP^{c}, is resistant to proteinase-K-digestion. It appears, therefore, that PrP^{Sc} is formed post-translationally from PrP^{c}. The illness arises and develops, depending upon the advance of the transformation of PrP^{c} into PrP^{Sc}. The factors which are answerable for the conversion of PrP^{c} to PrP^{Sc} are not yet fully known.

All previous attempts to develop a TSE therapy have directed the investigations to substances which are targeted to specifically bind to PrP^{Sc} or which block binding to PrP^{c} as is described in the publication C. Soto Biochem. Soc. Trans. 2002 August 30 (4): 569-574 which collects the various works on this point.

It is thus the object of the invention to provide a pharmaceutically effective agent and an alternative method which can treat TSE pathology, can make the illness less severe or can act preventatively.

Surprisingly, it has been found that a reduction in PrP^{Sc} formation and even an inhibition thereof can be obtained when prion infected cells of certain peptides are supplied. It has been found that with the peptides according to the invention which have PrP^{c} accumulated therein, the conversion of the naturally available PrP^{c} into the pathogenic PrP^{Sc} is prevented. According to the invention, various peptides with partly characterized amino acid-sub-sequence blockers have been identified by which an amelioration of the symptoms or even a healing of the TSE pathology can occur. Exemplary peptides are given in the sequences 1 through 27. As peptides which can be used according to the invention, the following sequences which have been identified and which are to be read in the direction from the amino terminal to the carboxyl terminal and which can be used as one component or in a mixture of at least two components for the treatment:

All sequences which form at least one component from at least one of the groups comprised of:

A) Val and Asp, Met and Ile, Asp and Val, Gln and
Pro, Thr and Pro, Leu and Asp, Asp and Ser, Arg
and His, Thr and Tyr, Val and Tyr, Arg and Pro,
Pro and Leu, Leu and Pro, Pro and Ser, Ser and
Pro, Leu and Lys, Lys and Ala, Ala and Thr, Thr
and Thr, Thr and Asn, Asn and Ser, Ser and Lys,
Lys and Leu, Leu and Met, Met and Met, Met and
Tyr, Trp and His, His and Trp, Trp and Gln, Gln
and Trp, Trp and Thr, Thr and Pro, Pro and Trp,
Trp and Ser, Ser and Ile, Ile and Gln, Gln and
Pro;
B) Leu and Asp and Ser, Val and Asp and Met, Asp
and Met and Ile, Met and Ile and Asn, Asp and Val
and Gln, Val and Gln and Pro, Gln and Pro and Leu,
Gln and Pro and Met, Pro and Leu and Thr, Leu and
Thr and Pro, Leu and Asp and Ser, Asp and Ser and
Ser, Asp and Ser and Cys, Arg and His and Ala, His
and Ala and Thr, Ala and Thr and Tyr, Val and Tyr
and Ser, Tyr and Ser and Ser, Arg and Pro and Leu,
Pro and Leu and Pro, Leu and Pro and Ser, Pro and
Ser and Pro, Leu and Lys and Ala, Lys and Ala and
Thr, Ala and Thr and Thr, Thr and Thr and Asn, Thr
and Asn and Ser, Asn and Ser and Lys, Ser and Lys
and Leu, Lys and Leu and Met, Leu and Met and Met,
Met and Met and Tyr, Trp and His and Trp, His and
Trp and Gln, Trp and Gln and Trp, Gln and Trp and
Thr, Trp and Thr and Pro, Thr and Pro and Trp, Pro
and Trp and Ser, Trp and Ser and Ile, Ser and Ile
and Gln, Ile and Gln and Pro;
C) Val and Asp and Met and Ile, Asp and Val and
Ile and Pro, Leu and Asp and Ser and Ser, Arg and
His and Ala and Tyr, His and Ala and Thr and Tyr,
Val and Tyr and Ser and Ser, Arg and Pro and Leu
and Pro, Pro and Leu and Pro and Ser, Leu and Pro
and Ser and Pro, Leu and Lys and Ala and Thr, Lys
and Ala and Thr and Thr, Ala and Thr and Thr and
Asn and Thr and Thr and Asn and Ser, Thr and Asn
and Ser and Lys, Asn and Ser and Lys and Leu, Ser
and Lys and Leu and Met, Lys and Leu and Met and
Met, Leu and Met and Met and Tyr, Trp and His and
Trp and Gln, His and Trp and Gln and Trp, Trp and
Gln and Trp and Thr, Gln and Trp and Thr and Pro,
Trp and Thr and Pro and Trp and Ser, Pro and Trp
and Ser and Ile, Trp and Ser and Ile and Gln, Ser
and Ile and Gln and Pro;
D) Val and Asp and Met and Ile and Asn, Asp and
Met and Ile and Asn and Asp, Met and Ile and Asn
and Asp and Val, Ile and Asn and Asp and Val and
Gln, Asn and Asp and Val and Gln and Pro, Asp and
Val and Gln and Pro and Leu, Val and Gln and Pro
and Leu and Thr, Gln and Pro and Leu and Thr and
Pro, Leu and Asp and Ser and Ser and Arg, Arg and
His and Ala and Thr and Tyr, Leu and Lys and Ala
and Thr and Thr, Lys and Ala and Thr and Thr and
Asn, Ala and Thr and Thr and Asn and Ser, Thr and
Thr and Asn and Ser and Lys, Thr and Asn and Ser
and Lys and Leu, Asn and Ser and Lys and Leu and
Met, Ser and Lys and Leu and Met and Met, Lys and
Leu and Met and Met and Tyr, Trp and His and Trp
and Gln and Trp, His and Trp and Gln and Trp and
Thr, Trp and Gln and Trp and Thr and Pro, Gln and
Trp and Thr and Pro and Trp, Trp and Thr and Pro
and Trp and Ser, Trp and Thr and Pro and Trp and
Ile, Thr and Pro and Trp and Ile and Gln, Pro and
Trp and Ile and Gln and Pro;
E) Val and Asp and Met and Ile and Asn and Asp,
Val and Gln and Pro and Leu and Thr and Pro, His
and Ser and Pro and Leu and Asp and Ser, Ser and
Arg and His and Ala and Thr and Tyr, Leu and Lys
and Ala and Thr and Thr and Asn, Lys and Ala and
Thr and Thr and Asn and Ser, Ala and Thr and Thr
and Asn and Ser and Lys, Thr and Thr and Asn and
Ser and Lys and Leu, Thr and Asn and Ser and Lys
and Leu and Met, Asn and Ser and Lys and Leu and
Met and Met, Ser and Lys and Leu and Met and Met
and Tyr, Trp and His and Trp and Gln and Trp and
Thr, Trp and Gln and Trp and Thr and Pro and Trp,
Trp and Thr and Thr and Pro and Trp and Ser and
Ile, Pro and Trp and Ser and Ile and Gln and Pro;
F) Asp and Val and Gln and Pro and Leu and Thr and
Pro, Leu and Asp and Ser and Ser and Arg and His
and Ala, Ser and Ser and Arg and His and Ala and
Thr and Tyr, Leu and Lys and Ala and Thr and Thr
and Asn and Ser, Lys and Ala and Thr and Thr and
Asn and Ser and Lys, Ala and Thr and Thr and Asn
and Ser and Lys and Leu, Thr and Thr and Asn and
Ser and Lys and Leu and Met, Thr and Asn and Ser
and Lys and Leu and Met and Met, Asn and Ser and
Lys and Leu and Met and Met and Tyr, Trp and His
and Trp and Gln and Trp and Thr and Pro, Gln and
Trp and Thr and Pro and Trp and Ser and Ile, Thr
and Pro and Trp and Ser and Ile and Gln and Pro;

G) At least two components of at least one of the groups A) to F);

H) Sequences 1 through 27 of the sequence protocol which comprise all 12 amino acids with the positions 1 through 12 or contain them.

These sequences can have an optional length, preferably a length of 6 to 40, especially preferably 8 to 30, 10 to 20 or even more preferred 10 to 12 amino acids long.

In several special embodiments, the following sequences are provided which are preferably 12 amino acids long;

I) Amino acid sequences which in the positions 1, 2, 3 and 4 contain the amino acids LEU, LYS, ALA and THR;

J) Amino acid sequences which in the positions 6, 7, 8 and 9 contain the amino acids Asn, Ser, Lys and Leu;

K) Amino acid sequences which are a combination of the features I) and J);

L) amino acid sequences which contain Gln, Trp and Thr in the positions 4, 5 and 6;

M) amino acid sequences which contain the amino acid Arg and His in the positions 8 and 9;

N) Amino acid sequences which contain the acids Thr and Tyr in the positions 11 and 12;

O) All subcombinations with two or three elements from the groups L), M) and N);

P) Amino acid sequences which contain amino acids Val and Tyl in the positions 1 and 2;

Q) Amino acid sequences which contain the acids Arg, Pro, Leu, Pro, Ser and Pro in the positions 7, 8, 9, 10, 11, 12;

R) amino acid sequences which are a combination of N) and O).

The sequence segments (I)-(R) can be contained in sequences which, for example have a length of 6 to 40 amino acids, preferably 8 to 30 amino acids, more preferably 10 to 20 amino acids and especially preferably 10 to 12 amino acids.

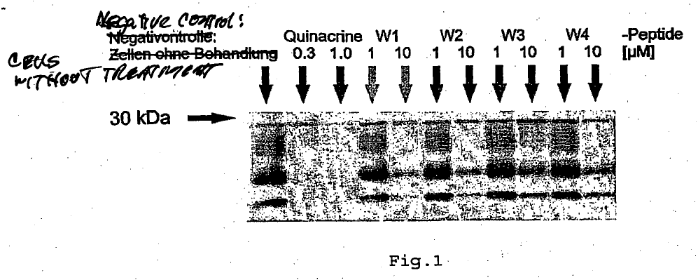

FIG. 1 shows the amino acid sequences (in single letter code) for the four effective peptides which have been identified in phage display screening

“W1” HSPLDSSRHATY = Seq. Nr. 8
“W2” VDMINDVQPLTP = Seq. Nr. 2
“W3” VYSSTTRPLPSP = Seq. Nr. 10
“W4” LKATTNSKLMMY = Seq. Nr. 1.

The peptides of the invention which are capable of curing or ameliorating TSE can be made by known methods.

Thus, for the production, a chemical-synthetic method, for example solid phase synthesis or a synthesis in liquid medium can be employed. In the solid phase synthesis the amino acids are bonded together in the sequence corresponding to the sequence protocols and the listing A)-R). The solid phase peptide synthesis is comprised of three important steps:

1) the build up of the amino acid chain to the peptide,

2) the splitting of the synthesized peptide from the resin; and

3) optional cleaning and characterization.

For the synthesis of the amino acid chains, different coupling methods are known as have been described for example in “Beyer Walter” 22. Vol. ISBN 3-7776-0485-2, pages 829 to 834.

The peptides can be made by expression of the nucleotide sequences coding for them, for example in chromisomes, plasmids or other information carriers, bare DNA or RNA in organisms, in cells or in cell free systems.

The subject of the invention thus includes also the nucleic acids which code for the peptides according to the amino acid sequences A)-R), including all allele variations as well as the nucleotide sequences of the sequence pattern 1 to 27 including all of their allele variations.

All mammals including humans can be treated with the peptides in accordance with the invention or cured and the peptides can also be used for prevention [prophylactively]. Especially preferred is the treatment of humans, although cattle and sheep can also be treated. The peptides according to the invention attach to the PrP^{c}. Of all mammals so that the effect of the invention can develop. The effect in accordance with the invention is also found when at least two of the mentioned peptides point to the PrP^{c}.

Using the peptides according to the invention a treatment of the TSE syndrome or illness is effected both with humans and with animals.

The human illnesses which can be treated include the Creutzfeldt-Jakob syndrome, Kuru, Gerstmann-Straussler-Scheinker syndrome and FFI (Fatal Familial Insomnia). With animals, the TSE illnesses which can be treated include, for example, scrapie in the case of sheep, bovine spongiform and encephatlopathy (BSE) and the wild animal, chronic wasting disease (CWD).

For the treatment the peptides according to the invention must be so applied that they reach the effective locations. The effective location is the brain, the spinal tissue and/or the entire nervous system as well as other parts of the organism. The peptides can be introduced in solid form or dissolved in a solvent, preferably water, to the body of the subject treated. As solids, the peptide can be administered for example orally, rectally or as a nasal powder. The effective substances according to the invention and pharmaceutical compositions containing same can have liquid, semiliquid solid or solid pharmaceutical forms and can be administered in the form, for example, of injectable solutions, drops, juices, sprays, suspensions, granulates, tablets, pellets, transdermal therapeutic systems, capsules, plasters, suppositories, salves, creams, lotions, gels, emulsions or aerosols and can be dispensed in such form as to contain the peptides of the invention in a physiologically tolerable form and together with pharmaceutical excipients depending upon the application method, like for example, carriers, fillers, solvents, diluents, surface active agents, cooling agents, preservatives, disbursing agents, lubricants, slide promoting agents, aromatizing agents and/or binders. Theses auxiliaries can for example be: water, ethanol, 2-propanol glycerine, fructose, lactose, saccharose, dextrose, molasses, starch modified starch, gelatin, sorbitol, inositol, mannitol, monocrystalline cellulose, methyl cellulose, carboxymethyl cellulose, cellulose acetate, shellac, cetlyalcohol, polyvinylpyrrolidone, paraffin, wax, natural and synthetic rubber, acacia gum, alginates, dextran, saturated and unsaturated fatty acids, stearic acid, magnesium stearate, zinc stearate, glyceryl stearate, sodium lauryl sulphate, edible oils, sesame oil, coconut oil, peanut oil, soybean oil, leciathin, sodium lactate, polyoxyethylene fatty acid esters and polyoxypropylene fatty acids esters, sorbitan fatty acid esters, sorbic acid, benzoic acid, citric acid, ascorbic acid, tannic acid, sodium chloride, potassium chloride, magnesium chloride, magnesium oxide, zinc oxide, silicon oxide, titanium oxide, titanium dioxide, magnesium sulfate, zinc sulfate, calcium sulfate, potash, calcium phosphate, dicalcium phosphate, potassium bromide, potassium iodide, talc, koalin, pectin, crosprovidone, agar and bentonite.

The choice of the auxiliary agent and the amount thereof which is used depends upon whether the medicament is applied orally, subcutaneously, parenterally, intravenously, pulmonarily, interperitoneally, transdermally, intramuscularly, nasally, bucally, rectally or in another appropriate way. For oral applications, formulations are suitable, among others, in the form of tablets, dragees, capsules, granulates, drops, juices and syrups. For parenteral administration, topical and inhalative applications, solutions, suspensions and easily reconstructable powders for inhalation as well as sprays can be used. In suitable percutaneous application forms, the effective material can be provided in a sustained or delayed release in soluble form or in a plaster, optionally together with skin penetration promoting agents. Rectal transmicosal, parenteral, oral or percutaneous formulations can liberate the peptides of the invention in a retarded or delayed form.

In its liquid form, the peptides according to the invention can be applied intravenously, orally, as nasal sprays, subcutaneously, intramuscularly, inhalationally or in adjacent spinal tissue. In addition, the effective material according to the invention can be applied by means of salves or creams.

The peptides according to the invention can be applied to the effective location or other locations of the organism based upon the nucleic acids (DNA and RNA) or a combination thereof which code for the peptides of the invention in the organism. This can be achieved through the use of viral vectors, bare nucleic acids (DNA, RNA), plasmids, synthetic virus particles and liposomes which are introduced intravenously, intranasally, orally, rectally, subcutaneously or intramuscularly, in or on the spinal cord or spinal cord marrow.

The peptides of the invention can also be used for prevention of the aforementioned pathologies [prophylactically]. In this case, the peptides of the invention must be so applied that they reach their effective locations. These effective locations can be the brain, spinal cord marrow and/or the entire nervous system, although any other part of the organism may be effected as well. For this purpose, the peptides of the invention are formulated in solid or in a solvent, preferably water, for administration in soluble form to the body. As solids, the peptides can be introduced orally, rectally or as nasal powder.

In liquid form, the peptides of the invention can be applied for example intravenously, orally, as nasal spray, subcutaneously, intramuscularly, inhalationally or in or adjacent the spinal cord. The effective material according to the invention can also be applied as salves or creams.

The peptides of the invention can be provided at the effective locations or other locations in the organism at which nucleic acids (DNA and RNA or a combination thereof) are present which code for the peptides of the invention in the organism. This can be achieved through the use of viral vectors, bare nucleic acids (DNA, RNA), plasmids, synthetic virus particles, liposomes, which can be applied intravenously, intranasally, orally, rectally, subcutaneously in or on the spinal cord in the body.

To improve the binding, solubilization and effectiveness properties, the peptides according to the invention can be modified so that the water solubility, stability, bioavailability and the affinity to adhesion of PrP^{c} is improved. These modifications can be effected for example by sugar residues, glucoronic acids, sulfate residues, serine, glycine or aspartate.

In addition, the peptides according to the invention can be linked with antibodies or fragments thereof in the usual way to activate parts of the immune system in the use of the proteins according to the invention.

As to the pathologies to be treated, Creutzfeldt-Jakob syndrome, Kuru, Gerstmann-Straussler-Scheinker syndrome and FFI (Fatal Familial Insomnia) can be named by way of example.

Methodology:

Phage display is a technique which enables peptide libraries to be constructed with randomized amino acid sequences and these to be researched with various ligands as to certain target molecules. The peptide library is thus presented as a fusion between a peptide and a-phage-sheath protein on the surface of bacterial phages (phage display). The diversity of the presented peptide is achieved by the insertion of a combinatorally mutated DNA as the peptide coding-part of the fusion gene. In this manner, an extremely large number of phages can be produced, whereby each phage presents another peptide. The construction, multiplication and selection are termed “biopanning”. The library is incubated with an immobilized target molecule, whereby the nonbinding portions of the peptide library are washed away and the binding part can then be eluted. The thus enriched population of phages, which presents a peptide capable of interacting with the target molecule, is amplified by using it to infect bacteria. The screening-amplification procedure can be repeated several times to further enrich the library participants which have a relatively high affinity to the target molecule. The result is a peptide population which is dominated by the amino acid sequences which best point to the target molecules.

Generally a commercial phage library is used with 12 randomized amino acids (New England Biolabs, Frankfurt, Germany). Here a gp3-gene coding for the phage sheath protein, depending upon the signal sequence N terminal of the library, is inserted. This is comprised of 1.9×10⁹ independent clones and is so amplified and concentrated that in 10 μl on the average, each sequence is present in 55 copies.

For carrying out the selection with respect to the recombinant prion protein (rPrP) from hamster, recombinantly produced hamster prion protein in a concentration between 0.01 μg/ml and 0.1 μg/ml in PBS with 0.2% SDS, PH 7.2 is immobilized with the aid of the Protein Immobilizer Kit (Exigon) in a microtiter plate recess. For this purpose, each 100 μl of protein solution is incubated for 2 hours under gentle shaking. After removal the solution is washed three times with 200 μl of PBS.

For the selection 10 μl of phages from the commercial phage library is incubated in 100 μl Pbst with 0.1% BSA for 10 minutes under light shaking in an rPrP coated microtiter plate recess. The nonbonded phages are discarded and then washing is carried out ten times with 300 μl PBST. The elution is carried out with 100 μl of 0.2 m glycene-HCL, pH 2.2 for ten minutes under light shaking. The eluate is neutralized in 15 μl Tris-HCL, pH 9.1.

The eluate is transferred to an E. coli culture and incubated for 4.5 hours at 37° C. and 200 rpm. Then the culture is centrifuged for 10 minutes at 5000 rpm. The supernatant is removed and 1/6 volumes of PEG/NaCl are added. The product is allowed to precipitate overnight at 4° C. The product is then centrifuged for 20 min at 5000 rpm and the supernatant is disgarded and then resuspended in 1 ml/PBs. Then the suspension is centrifuged for 5 minutes at 10,000 rpm. The supernatant is poured off and 1/6 volumes of PEG/NaCl are added. The composition is allowed to precipitate for one hour on ice. Then it is centrifuged for 60 minutes and the pellet resuspended in 100 μl/TBS. The thus obtained phages can be used for the next round. In total, five selection rounds are carried out. Then the phage clones which have been selected out are isolated and the amino acid sequences determined for the peptides presented on these phages by DNA sequencing of the coded peptides in the phage genome. The result is a total of the 27 different amino acid sequences given in the sequence protocol.

In a cell culture essay with infected N2a cells it was found that all of the investigated peptides were able to reduce the amount of PrP^{Sc} which was formed.

Example:

Test Description

Prion infected cells were cultured for a week and then investigated with respect to the formation of PrP^{Sc}. This was carried out in that the cells were lysed and treated with 20 μg/ml Proteinase-K (PK). As a result, PrPphu c was digested and only the PK resistant PrP^{Sc} remained. The PK treated cell lysate was subjected to a denaturing polyacrylamide gel electrophesis (SDS-PAGE) and blotted on a membrane and then stained with a PrP specific antibody (Western Blot). FIG. 1 shows the band pattern of the PK treated cell lysate. The peptides W1, W2, W3 and W4 corresponded to the sequences 8, 2, 10 and 1 according to the sequence protocol. The band pattern was typical (see trace 1 and the lower FIG.).

In the presence of substances which inhibit the multiplication of prions (Quinacrine as a positive control), there is no band pattern or only a clearly weak band pattern to be seen (traces 2, 3).

In the treatment of the prion infected N2a cells with peptides W1 to W4, there is a concentration dependent reduction in the PrP bands to be seen in the Western Blot (usual traces). This means that the peptides inhibited the prion replication effectively.

1. A peptide according to sequence number 1.

2. The peptide according to claim 1 characterized in that it is in solid, semiliquid or liquid form.

3. The peptide according to claim 1 characterized in that it is in the form of an injection solution, drop, juice, syrup, spray, suspension, granulate, tablet, pellet, transdermal therapeutic system, capsule, plaster, suppository, salve, cream, lotion, gel, emulsion or aerosol form.

4. The peptide according to claim 3 characterized in that it contains an auxiliary substance like, for example, a carrier, a filler, a solvent, a diluent, a surface active substance, a coloring agent, a preservative, a bursting agent, a smoothing agent, a lubricant, an aromatizing agent and/or a binder.

5. The peptide according to claim 1 characterized in that it is modified or substituted by at least one component from the group of sugar residues, glucoromic acid, sulfate residues, serine, glycine or aspartate.

6. A method of making an agent according to claim 1 characterized in that a solid phase synthesis in a liquid phase is used.

7. The method of making an agent according to claim 1 characterized in that a peptide according to claim 1 is expressed by a nucleic acid coding for this peptide sequence.

8. The use of the peptide according to claim 1 for producing a pharmaceutical.

9. A healing or treatment method as well as a method of preventing the illness TSE characterized in that the patient is enriched with a peptide according to claims 1 to 5.

10-12. (canceled)


SEQUENCE LISTING (27 sequences)

SEQ ID NO: 1 - protein, 12 aa, Artificial Sequence
  Description of the Artificial Sequence chemical synthesis
LKATTNSKLMMY

SEQ ID NO: 2 - protein, 12 aa, Artificial Sequence
  Description of the Artificial Sequence chemical synthesis
VDMINDVQPLTP

SEQ ID NO: 3 - protein, 12 aa, Artificial Sequence
  Description of the Artificial Sequence chemical synthesis
VDMIDDVQPLTP

SEQ ID NO: 4 - protein, 12 aa, Artificial Sequence
  Description of the Artificial Sequence chemical synthesis
VDMINDVQPMTP

SEQ ID NO: 5 - protein, 12 aa, Artificial Sequence
  Description of the Artificial Sequence chemical synthesis
VYMMNNGQPPSP

SEQ ID NO: 6 - protein, 12 aa, Artificial Sequence
  Description of the Artificial Sequence chemical synthesis
VDMINDVQPMSP

SEQ ID NO: 7 - protein, 12 aa, Artificial Sequence
  Description of the Artificial Sequence chemical synthesis
WHWQWTPWSIQP

SEQ ID NO: 8 - protein, 12 aa, Artificial Sequence
  Description of the Artificial Sequence chemical synthesis
HSPLDSSRHATY

SEQ ID NO: 9 - protein, 12 aa, Artificial Sequence
  Description of the Artificial Sequence chemical synthesis
HYTLDSCRHPTY

SEQ ID NO: 10 - protein, 12 aa, Artificial Sequence
  Description of the Artificial Sequence chemical synthesis
VYSSTTRPLPSP

SEQ ID NO: 11 - protein, 12 aa, Artificial Sequence
  Description of the Artificial Sequence chemical synthesis
VYSSNTRPLPSP

SEQ ID NO: 12 - protein, 12 aa, Artificial Sequence
  Description of the Artificial Sequence chemical synthesis
VYSSNNRPLPSP

SEQ ID NO: 13 - protein, 12 aa, Artificial Sequence
  Description of the Artificial Sequence chemical synthesis
VYLLNNRPLPSP

SEQ ID NO: 14 - protein, 12 aa, Artificial Sequence
  Description of the Artificial Sequence chemical synthesis
VYLLSTRPLPSP

SEQ ID NO: 15 - protein, 12 aa, Artificial Sequence
  Description of the Artificial Sequence chemical synthesis
VYWPTNRPLPSP

SEQ ID NO: 16 - protein, 12 aa, Artificial Sequence
  Description of the Artificial Sequence chemical synthesis
VQPSINRPHQRP

SEQ ID NO: 17 - protein, 12 aa, Artificial Sequence
  Description of the Artificial Sequence chemical synthesis
YHNYTTAPHSPS

SEQ ID NO: 18 - protein, 12 aa, Artificial Sequence
  Description of the Artificial Sequence chemical synthesis
KPVISPTNALQP

SEQ ID NO: 19 - protein, 12 aa, Artificial Sequence
  Description of the Artificial Sequence chemical synthesis
VTGPTKNLPATT

SEQ ID NO: 20 - protein, 12 aa, Artificial Sequence
  Description of the Artificial Sequence chemical synthesis
ASHVDYRRFLLT

SEQ ID NO: 21 - protein, 12 aa, Artificial Sequence
  Description of the Artificial Sequence chemical synthesis
DQDFAPDRHYRL

SEQ ID NO: 22 - protein, 12 aa, Artificial Sequence
  Description of the Artificial Sequence chemical synthesis
QKWPETYPDLSF

SEQ ID NO: 23 - protein, 12 aa, Artificial Sequence
  Description of the Artificial Sequence chemical synthesis
GDPVPQTYSAAG

SEQ ID NO: 24 - protein, 12 aa, Artificial Sequence
  Description of the Artificial Sequence chemical synthesis
AVSVNTKIDTEA

SEQ ID NO: 25 - protein, 12 aa, Artificial Sequence
  Description of the Artificial Sequence chemical synthese
QPNYTSLLYGTA

SEQ ID NO: 26 - protein, 12 aa, Artificial Sequence
  Description of the Artificial Sequence chemical synthesis
TQPPIHHYQLPA

SEQ ID NO: 27 - protein, 12 aa, Artificial Sequence
  Description of the Artificial Sequence chemical synthesis
GWDHIHGVHQHV